(19) United States
(12) Patent Application Publication
(10) Pub. No.: US 2023/0149928 A1
(43) Pub. Date: May 18, 2023

(54) SENSOR-BASED SYSTEMS AND METHODS FOR DIAGNOSTIC AND FIELD USE DETECTION OF NUCLEIC ACIDS

(71) Applicant: FemtoDx, Inc., Beverly Hills, CA (US)

(72) Inventors: David T. Weaver, Newton, MA (US); Robert DiNello, Alhambra, CA (US); Pritiraj Mohanty, Beverly Hills, CA (US)

(73) Assignee: FemtoDx, Inc., Beverly Hills, CA (US)

(21) Appl. No.: 17/916,633

(22) PCT Filed: Apr. 2, 2021
(86) PCT No.: PCT/US21/25568

§ 371 Date: Oct. 3, 2022

Related U.S. Application Data
(60) Provisional application No. 63/004,791, filed on Apr. 3, 2020.

(51) Int. Cl. B01L 3/00; C12Q 1/6844; B01L 7/00

(52) U.S. Cl. CPC B01L 3/502761; C12Q 1/6844; B01L 7/52; B01L 2300/0636; B01L 2200/16; B01L 2300/0877; B01L 2200/0652; B01L 2300/18

(57) ABSTRACT

Devices and methods for the detection of nucleic acids (e.g., RNA, DNA) are described. The nucleic acid can be obtained or derived from a pathogen, such as a virus. In one embodiment, the virus is a coronavirus (e.g., SARS-CoV-2) related to the disease COVID-19. Accordingly, devices and methods may be used in the field as point-of-care devices to test a subject (e.g., a patient) for the presence of the SARS-CoV-2 virus or another nucleic acid.

### RELATED APPLICATIONS

This application claims priority to U.S. Provisional Application No. 63/004,791, filed Apr. 3, 2020, which is incorporated herein by reference in its entirety.

### TECHNICAL FIELD

Devices and methods for detecting nucleic acids are generally described.

### BACKGROUND

Recent advances in genomics and proteomics can enable use of biomarkers to detect diseases at an early stage, predict optimal therapy tailor-made for specific patients and monitor therapy responsiveness. Despite the promise of biomarkers in screening, diagnosis and treatment, few biomarker-based tests are currently in clinical use. There can be a lag in the translation of biomarker research into clinically relevant tests, in spite of the potential impact of biomarker testing on cost effectiveness of detection and treatment, and on overall economic burden of care. Though this problem may arise due to technical, financial and regulatory challenges linked to the development and incorporation of biomarker testing into clinical practice, an important aspect of the problem seems to be the lack of a low-cost platform technology that can be employed at the point of care within the current clinical workflow.

### SUMMARY

Devices and methods for the detection of nucleic acids (e.g., RNA, DNA) are described. The nucleic acid can be obtained or derived from a pathogen, such as a virus. One example of a virus that can be detected is a coronavirus (e.g., SARS-CoV-2), which is a pathogen related to the disease COVID-19. Accordingly, devices and methods can be used in the field as point-of-care devices to test a subject (e.g., a patient) for the presence of the SARS-CoV-2 virus or another pathogen. The subject matter of the present invention involves, in some cases, interrelated products, alternative solutions to a particular problem, and/or a plurality of different uses of one or more systems and/or articles.

In one aspect, a device comprising a sample-receiving module; an amplification module, the amplification module comprising a plurality of isothermal amplification reagents; and a detection zone, the detection zone comprising; a sensor module comprising a sensor configured to detect a nucleic acid is described.

In another aspect, a method for detecting a target nucleic acid is described, the method comprising providing a sample comprising the target nucleic acid; heating the target nucleic to a temperature; exposing the target nucleic acid to a plurality of isothermal amplification reagents; amplifying the target nucleic acid to produce a plurality of amplified target nucleic acids; exposing at least a portion of the plurality of amplified target nucleic acids to a sensor configured to detect the amplified target nucleic acid; and detecting the target nucleic acid by a change in conductance.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1E are schematic diagrams of a device and method of amplifying a target nucleic acid within a sample, according to some embodiments;

FIG. 2A is a schematic of a target DNA strand with at least one zone for primer annealing, according to one set of embodiments;

FIG. 2B is a schematic diagram of a primer with a portion complementary to the target DNA and an extend portion from the 5′ end of the primer, according to some embodiments;

FIG. 2C is a schematic diagram of a first primer with a portion complementary to the target DNA and an extend portion from the 5′ end of the first primer and a second primer with a conjugated functional moiety attached to the 5′ of the second primer, according to one set of embodiments;

FIG. 2D is a schematic diagram of a first primer with a portion complementary to the target DNA and an extend portion from the 5′ end of the first primer and a second primer with a conjugated functional moiety attached to the 5′ of the second primer and a second conjugated functional moiety attached to the 3′ end of the primer, according to one set of embodiments;

FIGS. 3A-3B are schematic illustrations of an amplified target nucleic acid unbound and bound to a complementary nucleic acid portion of a sensor module, according to some embodiments;

FIGS. 3C-3D are schematic illustrations of a hairpin loop of nucleic acid with conjugated functional moieties on both ends of the nucleic acid conjugated to a sensor module whereby binding of the amplified target nucleic acid results in the hairpin loop opening, according to some embodiments;

FIG. 4 is a schematic illustration of a target DNA strand and different types of primers that can attach to the target DNA or to a nanosensor, according to certain embodiments;

FIG. 5A is as a schematic illustration of a nanowire functionalized with biomolecules to sense an analyte within a sample, according to some embodiments; and

FIG. 5B is a schematic of sensor module in communication with a reader module, according to certain embodiments.

### DETAILED DESCRIPTION

Devices and methods for detecting a nucleic acid (e.g., DNA, RNA) are generally described. The devices or the methods described herein can be portable, such as a handheld point-of-care device that can be administered by an external user, and the nucleic acid may be from, for example, a pathogen (e.g., a virus, a bacteria, a parasite) or other organism. As one example, devices and methods can be used to detect a coronavirus (e.g., SARS-CoV-2), which a virus that can cause the disease COVID-19. A sample (e.g., a nasopharyngeal swab, mucous, saliva, blood, urine, feces, a biological specimen) containing a target nucleic acid can be obtained from a subject (e.g., a person, a patient) and provided to the device or the method. Accordingly, devices and methods may be use detect the presence of the pathogen in the subject. As another example, the target nucleic acid can be the nucleic acid of a human such as DNA or RNA from a tumor or tumor cells, and devices and methods described herein can be used to detect the presence of cancer (e.g., a tumor, tumor cells) or monitor the disease and associated therapy. The target nucleic acid may be amplified under isothermal conditions using a plurality of amplification reagents (e.g., primers), which can increase the amount or the concentration of target nucleic acids. Upon amplification of the target nucleic acid, the amplified target nucleic acids may pass to a detection zone and/or a sensor (e.g., a nanosensor), where the sensor is configured to detect the target nucleic acid or the amplified target nucleic acid. The amplified target nucleic acid can be modified (e.g., possess additional nucleotides) relative to the target nucleic acid during the amplification process by selection of the appropriate amplification reagents. In some embodiments, the sensor is a nanosensor functionalized with a nucleic acid sequence that is complementary to the amplified target nucleic acid. Accordingly, the amplified target nucleic acid may bind to a nucleic acid sequence that is complementary to the amplified target nucleic acid and result in a change in conductance of the sensor in order to detect the target nucleic acid. The change of conductance of the nanosensor can be read by a reader module to send control and/or command signals of the device, such as reporting an output to the subject or an external user.

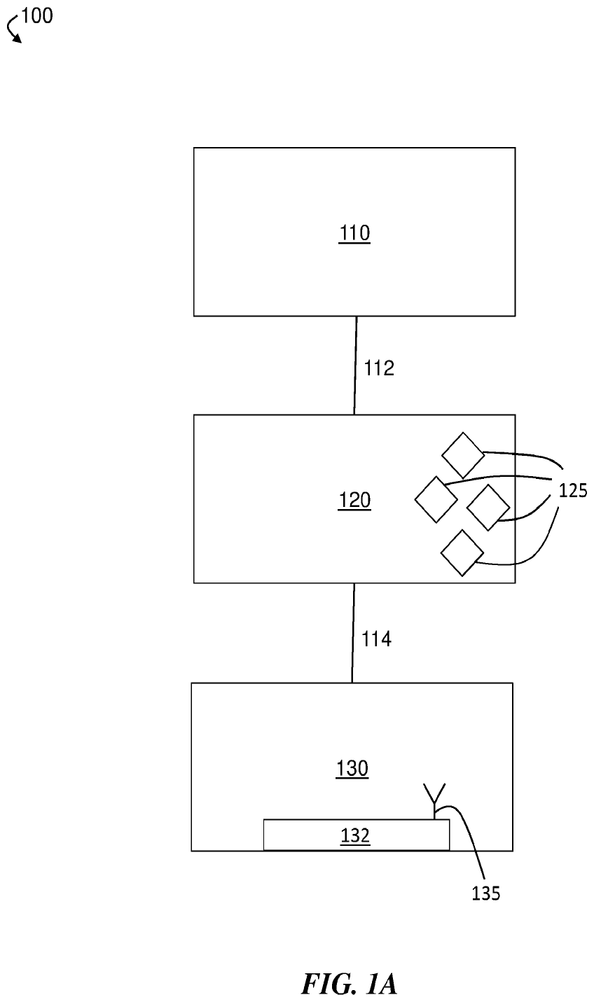

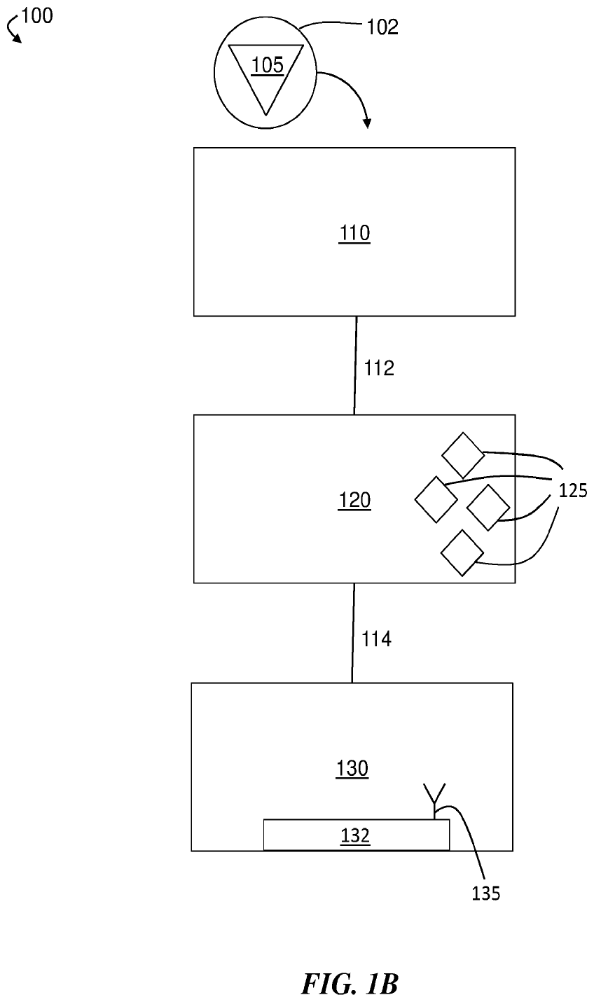

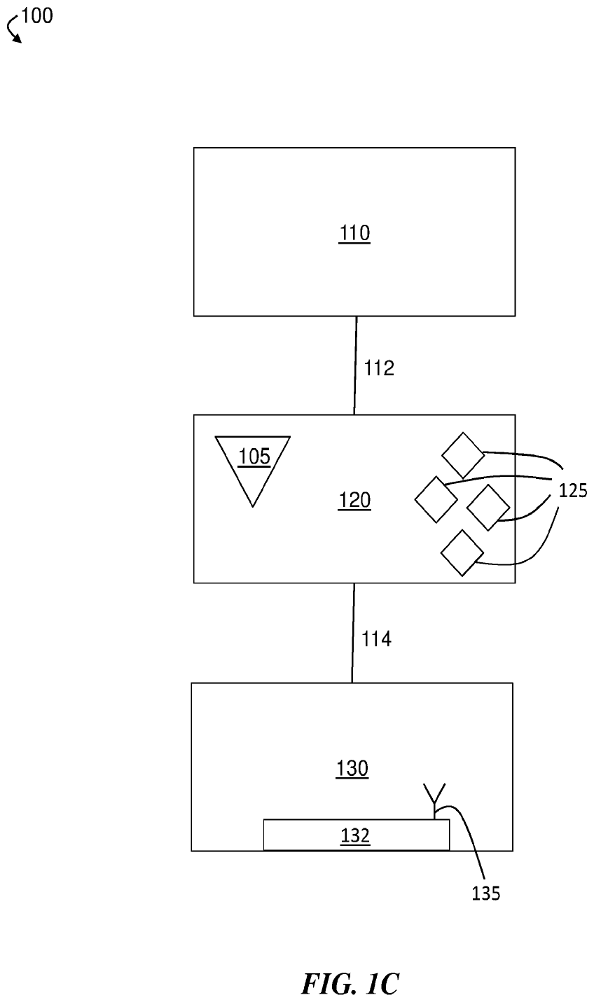

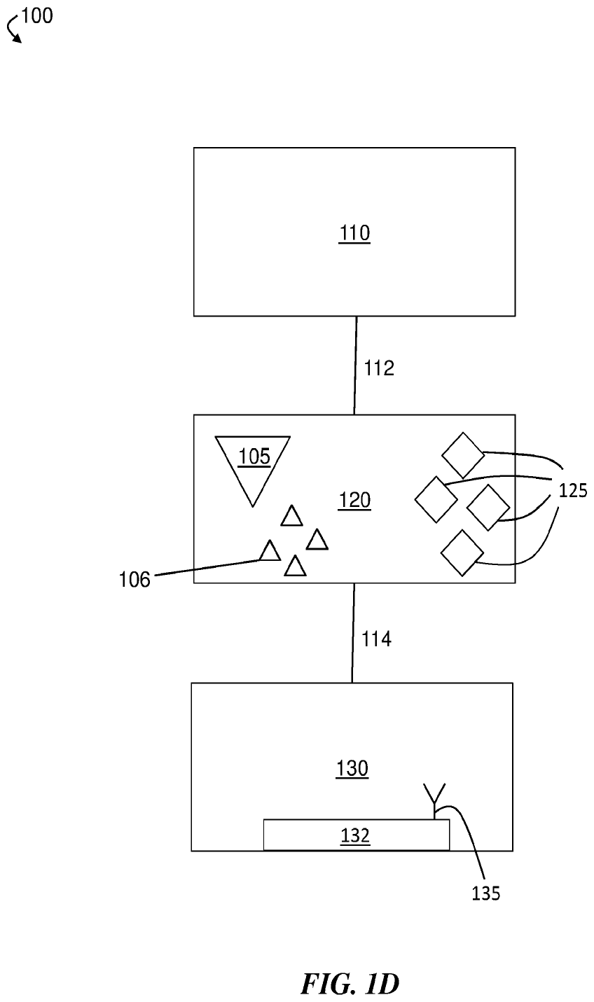

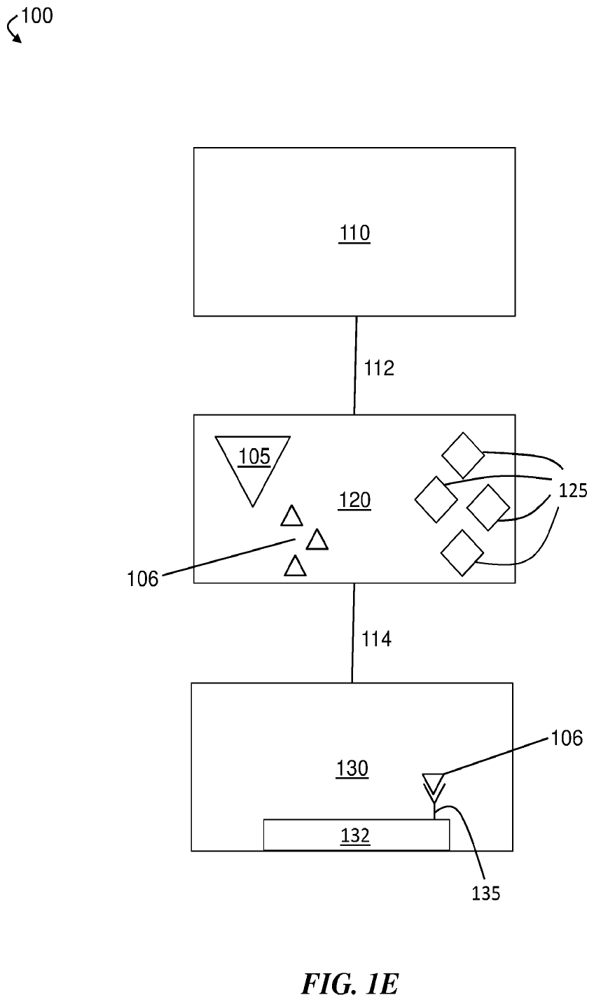

Certain embodiments comprise amplifying the target nucleic acid to produce a plurality of amplified target nucleic acids. Amplification of the target nucleic acid can occur in an amplification module comprising a plurality of isothermal amplification reagents (e.g., primers). For example, referring now to FIG. 1A, a device 100 comprises a sample receiving module 110, an amplification module 120 comprising a plurality of amplification reagents 125, a detection zone 130 with sensor module 132 comprising a complementary nucleic acid 135. In FIG. 1B, a sample 102 can comprise a target nucleic acid 120 and can enter the device through sample receiving module 110. Target nucleic acid 105 can be flowed into the amplification module 120, such as through channel 112, where amplification can take place, shown schematically in FIG. 1C. Certain embodiments comprise exposing the target nucleic acid to a plurality of isothermal amplification reagents. For example, in relation to FIG. 1C, target nucleic acid is exposed to plurality of isothermal amplification reagents 125. Some embodiments comprise amplifying the target nucleic acid to produce a plurality of target nucleic acids. An example of this is shown in FIG. 1D, where target nucleic acid 105 has been amplified to produce amplified target nucleic acids 106. In some embodiments, the amplified target nucleic acids may contain additional nucleotides (e.g., an extended 5′ portion) relative to the target nucleic acid. In some embodiments, the additional nucleotides are complementary to the nucleic acids of the sensor (e.g., the nanosensor). That is to say, in some embodiments, the amplified target nucleic acid may include an additional portion (e.g., an extended 5′ end) in order to facilitate detection and/or sensing. In some embodiments, the additional portion can bind to the sensor (e.g., the nanosensor).

Additional details regarding amplification are described below and elsewhere herein.

Some embodiments comprise detecting the target nucleic acid by a change in conductance. Detecting can occur in a detection zone. For example, in FIG. 1E, target nucleic acid 105 or at least one amplified target nucleic acid 106 can move from amplification module 120 to detection zone 130, for example, through channel 114 as shown in the figure. The detection zone can comprise a sensor module comprising a sensor configured to detect a nucleic acid. For example, in the figure, detection zone 130 can comprise a sensor module 132 configured to detect a nucleic acid, such as by using a complementary nucleic acid 135 attached to sensor module 132. Certain embodiments comprise exposing at least a portion of the plurality of target nucleic acids to a sensor configured to detect the target nucleic acid. In some embodiments, the complementary nucleic acid can attach to an amplified nucleic acid through a portion of the amplified nucleic acid, wherein the portion is not present in or complementary to the target nucleic acid. This additional portion can be added to the amplified target nucleic acid by an amplification reagent (e.g., an isothermal amplification reagent) during amplification of the target nucleic acid. In relation to FIG. 1E, sensor module 132 can be used to detect a nucleic acid such as amplified target nucleic 106 when exposed to complementary nucleic acid 135. That is to say, amplified target nucleic acid 106 can bind to a complementary nucleic acid 135. Attachment of amplified target nucleic acid 106 can cause a change in the surface charge or the conductance to sensor module 132.

Other components other than the sensor module can be present, such as valves, channels, or additional reagents that can facilitate in detecting a nucleic acid.

Sensing and/or detecting a nucleic acid by way of a change in surface charge or conductance to a sensor (e.g., a nanosensor) can provide several advantages. For example, a lower amount and/or concentration of nucleic acid (e.g., target nucleic acid, amplified target nucleic acid) may be needed when compared to existing systems. By contrast, certain existing systems detect a nucleic acid using spectroscopic methods (e.g., absorbance to certain wavelengths of light), which can require a relatively large amount and/or concentration of nucleic acid, which can increase the time needed to detect the nucleic acid. In addition, another advantage to devices and methods described herein using a change in conductance to sense and/or detect a nucleic acid is that the target nucleic acid can bind strongly to the complementary base pair of a sensor to provide a clear, strong signal, which can be used to clearly distinguish from the noise of a measurement, which can reduce erroneous measurements of the presence of a nucleic acid, such as the nucleic acid of a virus. As yet another advantage, the selectivity of the sensor (e.g., a complementary nucleotide of the surface of a sensor) and the sensitivity of the sensor can reduce the amount of analyte (e.g., amplification of the target nucleic acid) needed in order to detect the presence of the nucleic acid (e.g., the nucleic acid of a virus). Accordingly, the amount of amplified target nucleic acid or the degree of amplification may be considerably less than certain existing methods.

The detection zone can further comprise a reader module configured to send control and command signals to the sensor module to perform a task. For example, in relation to FIG. 1E, upon binding of amplified target nucleic acid 106 to sensor module 132 through complementary nucleic acid 135, the change in conductance can send a signal to the reader module (not pictured) in order to control or signal a sensor module or another component of device 100. In some embodiments, a communication channel is configured so that the reader module and the sensor module have a two-way communication.

In some embodiments, a sample-receiving module is associated with a device or a method. Accordingly, certain embodiments comprise providing a sample comprising the target nucleic acid. A schematic of such a sample-receiving module is shown in relation to FIG. 1A. Sample 102 can comprise target nucleic acid 105 and sample-receiving module 110 can provide sample 102 to the device or method. The sample-receiving module can be configured to receive a sample from a swab (e.g., a nasopharyngeal swab), saliva, mucous, or blood.

Certain embodiments comprise heating the target nucleic to a temperature. Heating may be provided in an amplification module, for example, amplification module 120 in FIGS. 1A-1E. The temperature can be maintained to create an isothermal environment for the target nucleic acid and/or the isothermal amplification reagents. “Isothermal” is given its usual meaning to refer to an environment where the temperature is constant throughout the environment. In some embodiments, the temperature is isothermally maintained at a temperature of at least 20° C., at least 25° C., at least 30° C., at least 35° C., at least 40° C., at least 45° C., at least 50° C., or at least 60° C., at least 70° C., at least 75° C., at least 80° C. or higher. In some embodiments, the temperature is isothermally maintained at temperature, no greater than 80° C., no greater than 75° C., no greater than 70° C., no greater than 60° C., no greater than 50° C., no greater than 45° C., no greater than 40° C., no greater than 35° C., no greater than 30° C., no greater than 25° C., or no greater than 20° C. or lower. Combinations of the above-referenced ranges are also possible (e.g., at least 40° C. and no greater than 55° C.). Other ranges are possible. Those skilled in the art are capable of selecting an appropriate isothermal temperature for a particular set of isothermal amplification reagents. The temperature can be heated, monitored, and/or maintained using any appropriate device. For example, a thermocouple can be used to heat, monitor, and/or the temperature. As another example, a thermometer can be used to monitor the temperature.

The heating can be provided to the amplification module to maintain an isothermal temperature for the amplification of a target nucleic acid. This may advantageously allow isothermal amplification of a target nucleic acid using isothermal amplification reagents. By contrast, certain existing devices and methods detect a nucleic acid (e.g., the nucleic acid of a virus) by using relatively high temperature thermocycling requiring high power and relatively long cycling times, which can reduce the portability of these certain existing devices and methods. In some cases using certain existing devices and methods, the use of an external laboratory is needed to detect a target nucleic acid, further lengthening detection times of these certain existing devices and methods.

However, as described herein, the use of isothermal amplification can provide lower energy requirements and can allow for device miniaturization, such that detecting of a nucleic acid (e.g., the nucleic acid of a virus) can be done relatively quickly. In some embodiments, detection of a nucleic acid can occur on a single chip (e.g., a micro-sized chip, a nano-sized chip) and without the need of an external laboratory. As another advantage, isothermal amplification reagents can comprise primers. Primers can be further functionalized, such as with an appropriate tag or molecular probe that can increase the selectivity of the amplified target nucleic acid. Additional details describing isothermal amplification and associated isothermal amplification reagents is provided below and elsewhere herein.

The following provides additional details regarding amplification, sensing, and detection.

In some devices and methods, there are at least two steps in the process: amplification and detection. Amplification is the process where a target nucleic acid (e.g., pathogen, virus, genomic DNA, genomic RNA) is increased in abundance, either by enzymatic routes or by non-enzymatic routes (e.g., thermal cycling). Some non-limiting examples of enzymatic routes are polymerase chain reaction (PCR) and a variety of isothermal DNA amplification methodologies. Isothermal DNA amplification methods include LAMP (i.e., loop-mediated isothermal amplification), rtLAMP (reverse transcription-loop-mediated isothermal amplification), RPA (i.e., recombinase polymerase amplification), NASBA (nucleic acid sequence-based amplification), MDA (multiple displacement amplification). Some other non-enzymatic routes include Hybridization Chain Reaction (HCR) and Catalytic Hairpin Assembly. Other amplification methods are possible.

In some embodiments, during amplification (e.g., isothermal DNA amplification, HCR), the concentration of target-specific nucleic acid increases approximately uniformly during the time of the reaction, assuming substrates are not limiting and product inhibition is not significant. With the example of PCR, the concentration of target-specific nucleic acid increases non-uniformly with cycle, but the cycle time abundance is proportional to concentration. In some embodiments, the device can be configured to contain an isothermal chamber with thermometer and temperature control operating in the range of 25-75° C. In some embodiments, the amplification module comprises the isothermal chamber.

In some embodiments, due to the sensitivity of the sensor (e.g., a nanosensor array detector), the amplification step does not need to be maximal. That is to say, amplification step may not require multiple rounds of amplification, especially when compared to certain existing systems. The amplified target nucleic acid may contain a portion of sequence not present or complementary to a sequence of the target nucleic acid, which may provide additional selectivity and/or sensitivity to the sensor such that additional amplification of the target nucleic may not be necessary in order to detect the target nucleic acid. Therefore, increased amplification conditions allow for secondary readings to confirm true negative samples (i.e., avoiding false detection of a nucleic acid) and allow for positive and negative controls to be incorporated.

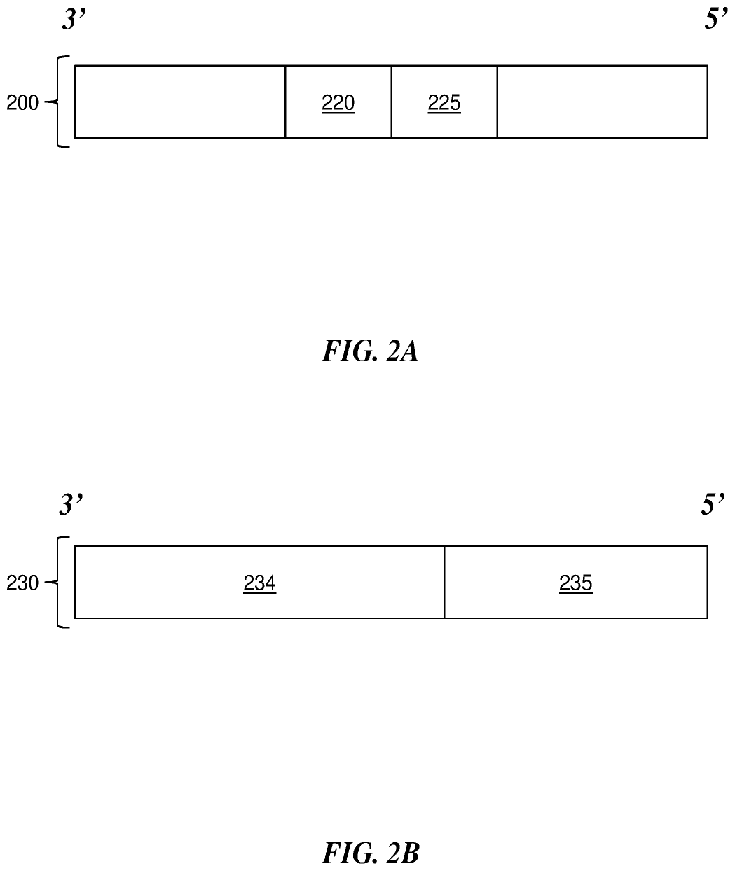

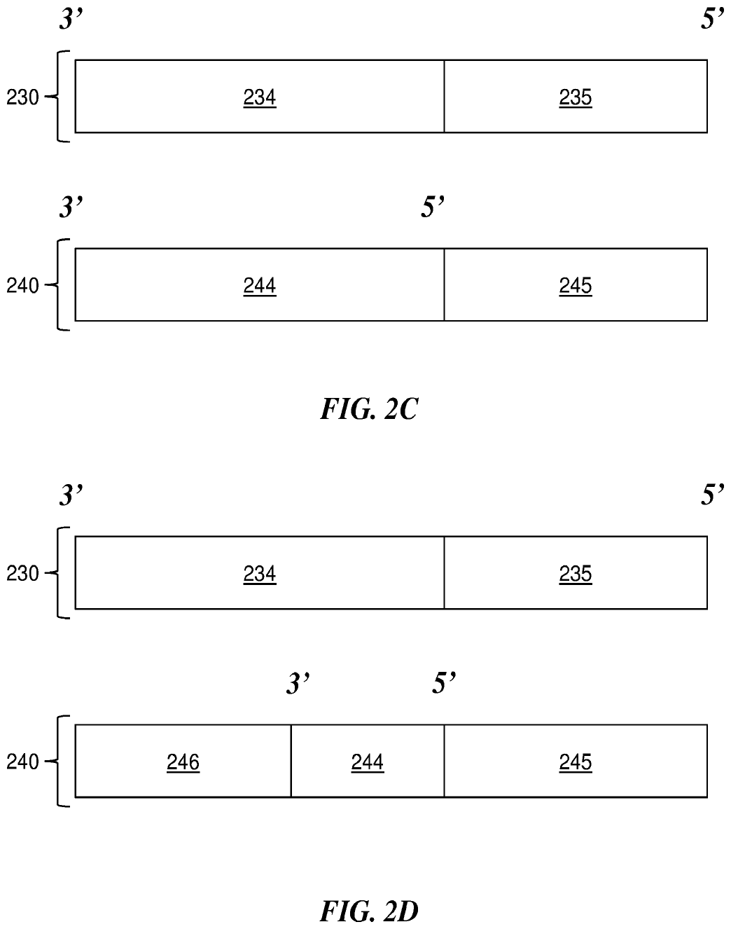

Primers can be used during amplification of a target nucleic acid. As used herein, “primers” are given their ordinary meaning in the art to refer to a short nucleic acid sequence (e.g., 18-22 basepairs) that provides a starting point for the synthesis of amplified nucleic acid (e.g., DNA synthesis amplified target nucleic acid). The primers can attach to certain complementary zones of the target nucleic acid. For example, in FIG. 2A, a target nucleic acid, such as target DNA 200, can have zones 220 and 225 that can anneal (i.e., complementarily bind) to primers. In some embodiments, one or more of primers has a 5′ extended region for basepairing (e.g., complementary base pairing) to an oligonucleotide (e.g., a complementary nucleic acid) on the nanosensor detector. For example, in relation to FIG. 2B, a primer 230 comprises complementary portion 234 that is complementary to the target nucleic acid, and an extended 5′ portion 235 that is not complementary to the target nucleic acid, but can be complementary to a nucleic acid sequence (e.g., an oligonucleotide) of the sensor module (e.g., the nanosensor detector). In some embodiments, the 5′ end of the primer is extended by at least 5 base pairs, at least 10 base pairs, at least 15 base pairs, at least 20 base pairs, at least 30 base pairs, at least 40 base pairs, or at least 50 base pairs or more. Other ranges are possible. In such embodiments, the 5′-extended region would not be recognized by the target nucleic acid during amplification. That is to say, the target nucleic acid would not be complementary to the extended 5′ region of the primer, but could be recognized by basepairing to an oligonucleotide conjugated to the sensor (e.g., a nanosensor) during detection and/or sensing. Without wishing to be bound by any theory, because the primer is used in isothermal amplification, the concentration of the primer associated with the amplified region can increase along with amplification of the target nucleic acid. Other modifications to the primer are possible, such that the amplified target nucleic acid can have increased selectivity for the sensor relative to the target nucleic acid (e.g., target DNA).

In some embodiments, one or more of the primers have a 5′ extended region for complementary basepairing to an oligonucleotide on the sensor (e.g., a nanosensor) detector, and a second primer has an extended 5′ region for conjugation to a molecular probe. For example, referring to FIG. 2C, primer 230 comprises extended 5′ portion 235 and primer 240 comprises conjugated functional moiety 245 that can be used for attachment to a molecular probe. The molecular probe can increase the gain of the signal or the conductance change received by the sensor when the amplified target nucleic acid binds to the sensor. In certain embodiments, an oligonucleotide hairpin configuration is used as the second primer, where both 5′ and 3′ ends may be conjugated to separate molecular probes. For example, as schematically illustrated in FIG. 2D, primer 240 now comprises two functional conjugated moieties, conjugated functional moiety 245 on the 5′ end of the primer, and conjugated functional moiety 246 on the 3′ end of the primer. In some embodiments, the molecular probe comprises nanoparticles. The nanoparticles can be of a defined size (e.g., cross-section diameter) such as 2 nm in cross-sectional diameter. In one embodiment, the molecular probe comprises a gold nanoparticle. Other molecular probes are possible. Additional non-limiting examples of molecular probes include small molecules (e.g., no greater than 500 g/mol, no greater than 750 g/mol, no greater than 1000 g/mol), quantum dots, small clusters (e.g., metallic clusters), proteins, biomarkers, molecular tags, active pharmaceutical ingredients (APIs), and/or fluorescent probes. The addition of a molecular probe may enhance the sensitivity or selectivity of the amplified target nucleic acid relative to target nucleic acid.

In some embodiments, the primer nucleotides are derivatized. Derivatization can be made on the ribose sugar of the nucleotide, on the phosphate of the nucleotide, and/or on the base of the nucleotide. For example, derivatization can be made to comprise a molecular tag, as described above. As another example, derivatization of the nucleotide can include the addition or modification of an alkyl (e.g., a cycloalkyl), a heteroalkyl, an aryl, a heteroaryl, an acyl, and/or an azide. Other derivatizations are possible. In some embodiments, the primer nucleotides are derivatized to form a specific charge configuration. In such embodiments, the specific charge configuration can increase or enhance sensing and/or detecting at the sensor (e.g., the nanosensor). Those skilled in the art are capable of selecting appropriate derivatives to enhance the charge configuration in the context of the teachings of this disclosure.

As described above and elsewhere herein and without being bound by any particular theory, detection (e.g., sensing) may be achieved by promoting alterations in the Debye length distribution of a molecular probe (e.g., a complementary nucleic acid) near or attached to the surface of the sensor (e.g., a semiconductor nanosensor) either directly or indirectly, causing a conductance change, and which subsequently converts to an electrical signal. In some embodiments, a reader module can receive or transmit the electrical signal.

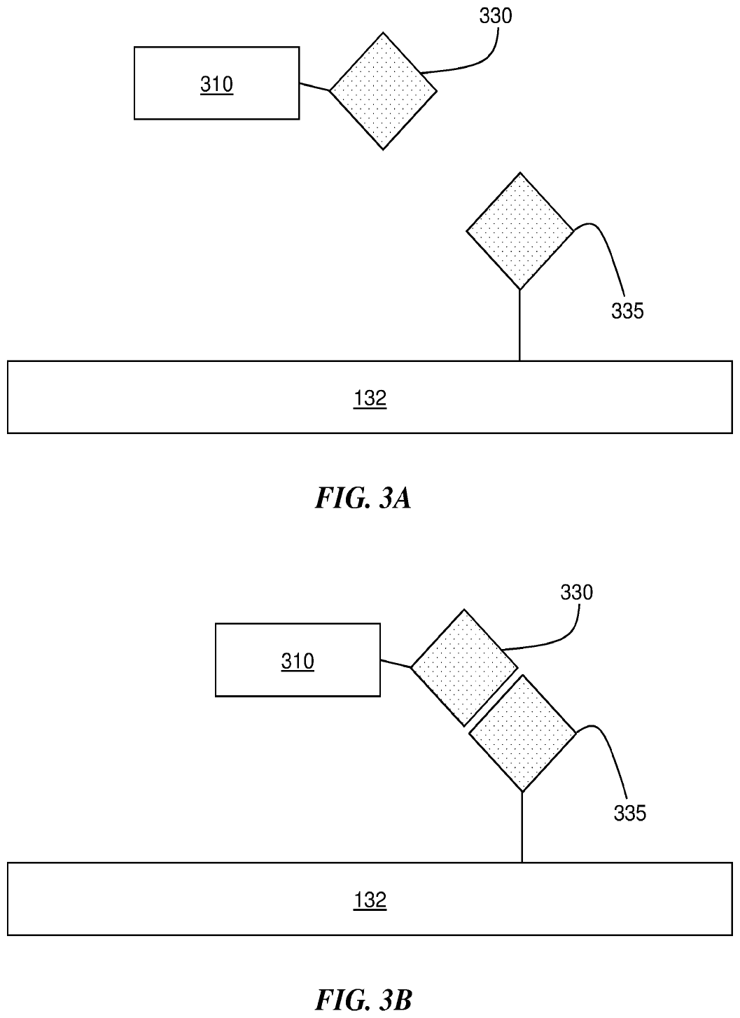

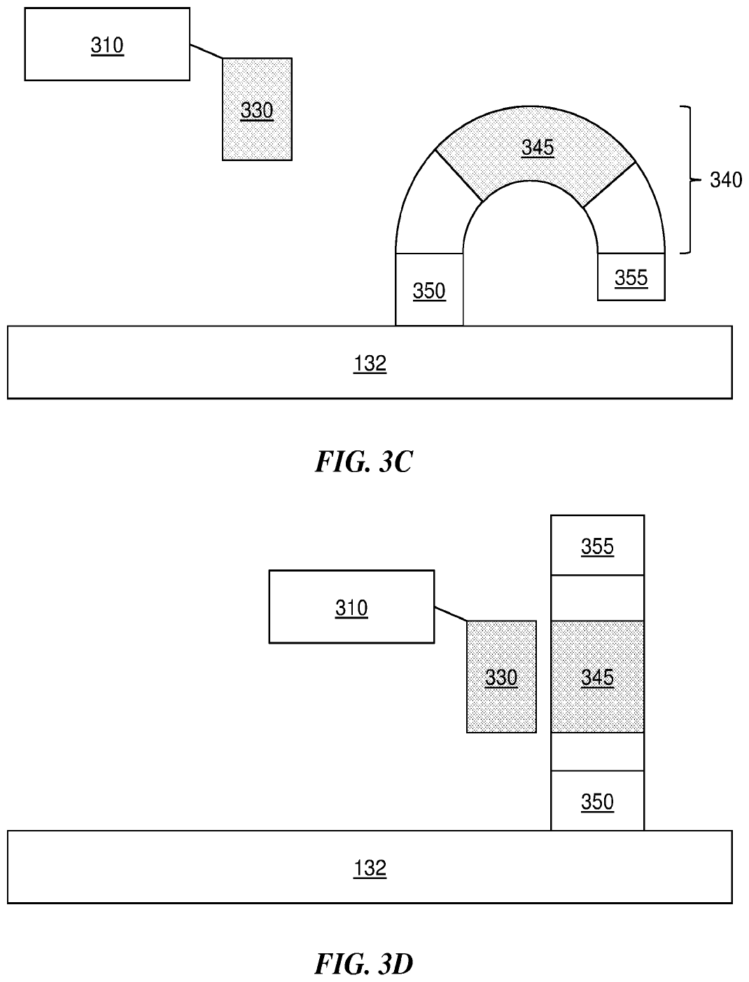

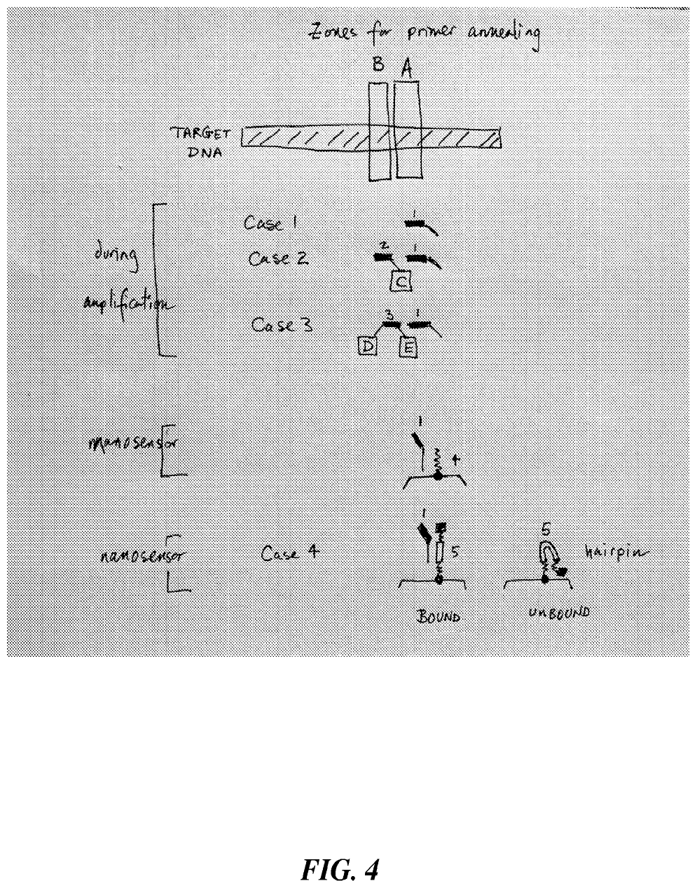

Detection for the sensor (e.g., the nanosensor) of the device or method can utilize an oligonucleotide hybridization schema where the product of isothermal amplification is evaluated. In this manner, an oligonucleotide receptor (e.g., a complementary nucleic acid) can be conjugated to the sensor surface. The oligonucleotide can be configured to provide complementary basepair recognition in one portion of the oligonucleotide and a linker attachment to the sensor surface in another portion of the oligonucleotide. For example, in relation to FIG. 3A, an amplified target nucleic acid 310 comprises an extended 5′ portion 330 that can bind to a complementary nucleic acid 335 of the sensor module 132. Binding to the sensor module can alter the surface charge of the sensor or a change in the conductance to produce a signal. Optionally, PNAs (i.e., peptide nucleic acid) or other derivatives of oligonucleotides such as those containing modified nucleotides can provide specifically tunability, such as specialized chemical properties (e.g., enhanced conduction, luminescence), and aptamers may be used for receptor interactions. Without wishing to be bound by any theory, due to the strength of basepairing between a primer with an extended 5′ end and the sensor detector (e.g., nanosensor detector) with complementary basepairing to the extended 5′ end of a primer, the ionic conditions for detection can be specifically tuned.

In some embodiments, a hairpin single-strand oligonucleotide is conjugated to the sensor (e.g., nanosensor) surface. The hairpin composition can be such that the termini of the oligonucleotide are conjugated to entities that contribute to the electric charge and its distribution relative to the sensor. For example, as schematically depicted in FIG. 3C, a hairpin loop 340 is conjugated with functional moieties 350 and 355. In certain embodiments, the hairpin is of 25-75 nucleotides in length (e.g., at least 25 nucleotides in length and/or no greater than 75 nucleotides in length) and contains self-complementary regions on either the 5′ end or the 3′ end or both of 10-20 nucleotides (e.g., at least 10 nucleotides in length and/or no greater than 20 nucleotides in length). Other lengths of the nucleotides are possible.

Upon annealing of the hairpin to the target sequence (e.g., the target nucleic acid, amplified nucleic acids), the separation of the two charged entities on the 5′ and 3′ ends and/or the redistribution of the charge delivers a change in Debye length at or near the sensor surface, and a corresponding change in conductance can be measured. For example, in FIG. 3D, complementary nucleic acid 345 of the hairpin loop 340 can anneal with extend 5′ portion 330 of the amplified target nucleic acid 310. Conjugated functional moiety 355 is moved away from the surface of sensor module 132, which may aid in sensing a nucleic acid by causing a redistribution of the charge or a modification of the Debye length. Without wishing to be bound by any theory, due to the strength of hairpin self-annealing under stringent ionic conditions, the ionic conditions for detection can be specifically tuned.

In some embodiments, detection on the device, optionally, does not use an oligonucleotide receptor, but uses the formation of a product of DNA amplification where the product comprises a proton (i.e., H⁺) or a hydronium ion (H3O⁺), and the sensor (e.g., the nanosensor) senses the concentration of protons/hydronium ions (acidity). In some embodiments, the detection method or detection by the device may include the enzymatic addition of a nucleotide on a DNA and/or RNA template-directed manner so that the nucleotide addition is measured in a quantitative manner.

Amplification and detection can occur in a coupled manner such that sensor (e.g., the nanosensor) reads may be continuous or discontinuously recorded. That is to say, in some embodiments, amplification and detection can occur in tandem. Coupling of the two parts may occur by microfluidic switches between chambers containing the amplification and the detection modules and/or components. However, in other embodiments, amplification and detection are uncoupled, and can also be on physically separate devices. In such an embodiment, amplification may occur in a cartridge, and the cartridge can be inserted into the device for reading. The cartridge and the device may be connected by snapping mechanical force and/or electrical components between the cartridge and the device, as a means to introduce the amplification product into a microfluidic chamber to direct the amplified target nucleic acid to the sensor (e.g., the nanosensor) surface.

In some embodiments, a sensor module is used to sense or detect an analyte (e.g., a target nucleic acid, an amplified target nucleic acid. The presence of an analyte (e.g., a biomarker) can be detected by a nanoscale field-effect transistor sensor through the measurement of conductance change of bio-functionalized nanowires (e.g., nanowires functionalized with a complementary nucleic acid). These sensors can serve as fundamental building blocks of a sensor module. Without wishing to be bound by any theory, the change in conductance is primarily due to the contribution of surface (charge) states to the conductance, which for larger sensors is dominated by volume effects. The fractional change is greatest for the smallest sensors, due to the increased surface-to-volume ratio. The presence of charged analytes (e.g., proteins, nucleic acids) on the surface of an active nanowire induces a large fractional change in the nanowire conductance, and enables relatively easy detection.

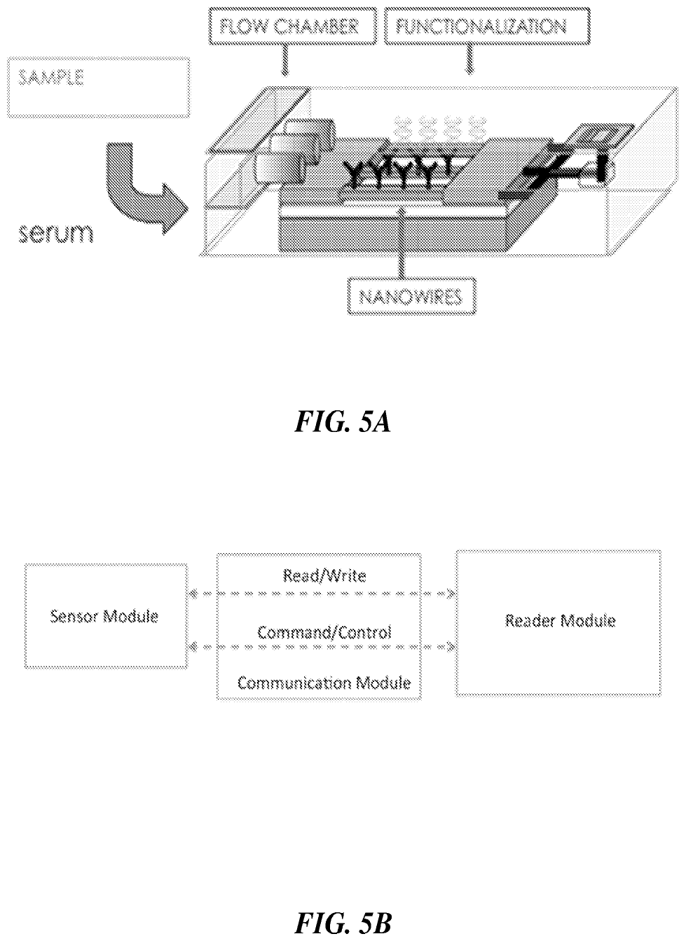

In some embodiments, the sensor comprises a nanoscale ion-sensitive FET (field effect transistor), fabricated with a “bottom-up” method, has been shown to be an effective way to monitor the concentration of chemical and biological entities (e.g., proteins, nucleic acids) in the solution by detecting the changes in the surface potential, due to either point charges or dipoles associated with biomolecules. In contrast, in some embodiments, a “top-down” method for device fabrication is employed and can create a nanoscale biosensor with complete control of the geometry, allowing for operation under conditions of controlled bias. The geometry and alignment of the nanowire can be fully controlled by lithography and standard semiconductor processing techniques in a CMOS-compatible process. The silicon nanowires are fabricated from silicon-on-insulator (SOI) wafer by electron beam lithography and surface nanomachining, which provide highly controllable nanowire sensors in comparison to other nanoelectronic approaches. FIG. 5A shows a schematic of a nanowires functionalized with a nucleic acid in order to detect a sample.

Suitable sensors have also been described in U.S. Publication No. US 2019/0094174, filed Jun., 2, 2017, and entitled “DEBYE LENGTH MODULATION,” which is incorporated herein by reference in its entirety for all purposes.

In some embodiments, a reader module configured with communication between the sensor module. The reader module can be configured to send and/or receive controls and commands to the sensor module, for example, to perform the necessary task, sending additional information such as calibration data for on-chip data processing, reading out the sensor data and sharing data. The communication channel can be either wired or wireless, so that the reader (e.g., the reader module) and the sensor (e.g., the sensor module) can have a two-way communication. A schematic example of the reader module in communication with the sensor module is shown in FIG. 5B.

The reader module may also contain necessary electronics to share or receive data and command/control sequence with another system capable of performing analytics. This system can be a cloud computer or a network that may communicate with the reader through either wired or wireless communication. The analytics performed by this system may provide an actionable decision or support data and analysis for an external user (e.g., a clinician, a healthcare professional) to make that decision (e.g., a diagnosis of a condition).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A device, comprising:
a sample-receiving module;
an amplification module, the amplification module comprising:
a plurality of isothermal amplification reagents; and
a detection zone, the detection zone comprising;
a sensor module comprising a sensor configured to detect a nucleic acid;

2. A method for detecting a target nucleic acid, the method comprising:
providing a sample comprising the target nucleic acid;
heating the target nucleic to a temperature;
exposing the target nucleic acid to a plurality of isothermal amplification reagents;
amplifying the target nucleic acid to produce a plurality of amplified target nucleic acids;
exposing at least a portion of the plurality of amplified target nucleic acids to a sensor configured to detect the amplified target nucleic acid; and
detecting the target nucleic acid by a change in conductance.

3. The device or method of any one of the preceding claims, wherein the target nucleic acid comprises coronavirus DNA and/or RNA.

4. The device or method of any one of the preceding claims, wherein the plurality of isothermal amplification reagents comprises isothermal primers.

5. The device or method of any one of the preceding claims, wherein the amplified target nucleic acid comprises a portion of a nucleotide sequence that is neither identical nor complementary to the target nucleic acid.
